(19) United States
(12) Patent Application Publication
(10) Pub. No.: US 2021/0139947 A1
(43) Pub. Date: May 13, 2021

(54) METHOD FOR ASSESSING SUSCEPTIBILITY OF A VIRUS TO TREATMENT BY MEASURING ENZYME ACTIVITY AND A SYSTEM THEREFORE

(71) Applicant: Cavidi AB, Uppsala (SE)

(72) Inventors: Stina Enqvist, Uppsala (SE); Clas Källander, Herräng (SE); Tommy Gatu, Uppsala (SE); Anders Malmsten, Uppsala (SE); Ingvar Pettersson, Uppsala (SE)

(21) Appl. No.: 16/628,220

(22) PCT Filed: Jul. 3, 2018
(86) PCT No.: PCT/SE2018/050726

§ 371 Date: Jan. 2, 2020

(30) Foreign Application Priority Data
Jul. 4, 2017 (SE) 1750874-8

(51) Int. Cl. C12Q 1/48; C12Q 1/02

(52) U.S. Cl. CPC C12Q 1/485; G01N 2333/15; G01N 2333/9128; C12Q 1/025

(57) ABSTRACT

The present invention relates to a method for assessing susceptibility of a virus to treatment with a drug inhibiting an enzyme of the wild-type virus comprising the steps: a) extracting the viral enzyme from a sample containing the virus; b) measuring the viral enzyme activity both in the absence of the drug and in the presence of the drug at a single predetermined concentration; and c) determining the virus' susceptibility to treatment with the drug from the relation between the enzyme activities in the presence of the drug and the absence of the drug. The invention further relates to a system for performing the method.

### FIELD OF THE INVENTION

The present invention relates to biological assays and in particular a method for determining the viral drug susceptibility of a virus strain infecting an individual and the viral load in the individual. The invention also relates to a system for performing such methods.

### BACKGROUND

The principle aim of anti-retroviral therapy (ART) is durable suppression of replicating plasma virus to undetectable levels, thereby delaying disease progression and prolonging survival. Treatment of HIV-1 in resource-limited settings currently relies on a combination of two nucleosides with a non-nucleoside reverse transcriptase inhibitor (NNRTI), either nevirapine (NVP) or efavirenz (EFV) as first-line treatment (Neogi et al 2013). Expanding access to ART along with close monitoring is needed for successful treatment outcomes. In high-income settings, this is achieved by performing quantitative viral load monitoring every 3-6 months as viral load monitoring detects treatment failure (Bryant 2013). Early detection of viral-failure provides the opportunity to intensify adherence counseling to improve adherence to ART potentially leading to re-suppression of viral load before the evolution of drug resistant virus can take place.

The currently accepted marker for viral load is measurement of HIV RNA in plasma. This can be accomplished either by PCR, NASBA, or by branched DNA techniques (See Revets et al 1996). All these assays are based on the amplification of HIV-1 virion RNA, which is considered impractical for wide-scale use in resource-limited settings, as it requires infrastructure, facilities for molecular diagnostics, expensive equipment and skilled technicians which are often unavailable.

An alternative to measure the HIV-1 RNA is to measure the activity of the viral reverse transcriptase (RT) enzyme. Cavidi, AB, Sweden have developed a technically less-demanding assay using an enzyme linked immune sorbent assay (ELISA)-based method to measure RT enzyme activity that has shown promising results (Labbett 2009, Huang 2010, Gupta 2016). The procedure used consists of the following steps: I) Inactivation of host polymerase activity that is present in the sample without affecting the viral enzymes present in the enveloped virion (PCT/SE002/00612). II) Removal of the enzyme-inactivating agent, enzyme activity blocking antibodies, endogenous enzyme activity inhibitors and antiviral drugs. III) Extracting the concentrated purified viral RT (PCT/SE01/00617). IV) Quantifying the RT activity using a sensitive enzyme assay (PCT/EP00/05563).

The selection of HIV-1 drug resistance among those failing first-line ART limits second-line and future treatment options. Antiretroviral drug resistance testing is thus important in long time management of HIV-1 infection. As traditional phenotypic resistance assays are time consuming, expensive and require specialized laboratory facilities, genotypic resistance testing is commonly used. However, many barriers exist for the use of genotypic resistance testing in low-income countries, such as the costs and the requirement of specific equipment. Therefore, other methods are sought for. One alternative is to determine the phenotypic virus susceptibility at the RT enzyme level. The ideal situation is to characterize enzyme that has been extracted directly from the virus circulating in the blood of the patient. The benefit of such an approach is that the sample will mirror the virus population replicating in the patient at the time the blood sample was taken.

Failure of first-line antiretroviral treatment (ART) among 10-30% of treated patients may be due to transmitted or acquired drug resistance, drug toxicity, reduced adherence and treatment interruptions (Goodall et al 2014, Hamers 2012 a,b). Analyze of phenotypical susceptibility to RT inhibitory drugs is thus potentially an affordable test to timely differentiate between drug resistance and reduced adherence. The concept has been explored by resistance testing with the PCR based Amp RT assay (US581745, U.S. Pat. No. 5,849,494).

Another assay based on a different line of technical development is the drug susceptibility assay developed by Cavidi AB. This procedure performs drug resistance testing on enzyme that is extracted directly from patient plasma samples. It is based on similar methods as used in the Cavidi HIV VL assay mentioned above. The virus is purified by ion exchange chromatography and the drug sensitivity profile of extracted viral enzyme is characterized by determination of IC₅₀ values in sensitive enzyme assays (PCT/SE02/01156, Shao et al 2003).

Although potentially useful these methods has so far been of limited practical importance in the management of the HIV pandemic. The Amp RT assay is quite sensitive but in best case semi quantitative and it is also hard to design controls to take the irregularities occurring during the initial reverse transcription into account. The drawbacks of the Cavidi methods are that they are labor intensive and have a long turnaround time. The need to analyze each extracted enzyme in presence of several drug dilutions also increase the total amount of enzyme required and compromise the test sensitivity.

### SUMMARY OF THE INVENTION

The current invention is a technically simple robust test providing integrated information about both HIV viral load (VL) and susceptibility to antiretroviral treatment (ART). The assay is adapted for automation, the turnaround time is reduced and each extracted enzyme sample only has to be analyzed in presence of one predetermined drug concentration.

The present invention is based on the idea that in order to assess drug susceptibility for a patient infected with a virus, such as HIV, and treated with a drug inhibiting an enzyme produced by the infecting virus, it is possible to determine the activity of the enzyme in the absence of the drug and in the presence of the drug at a single predetermined drug concentration, and comparing the enzyme activities in the presence and absence of drug, respectively, in order to obtain an assessment of the drug susceptibility. This is an advantage in relation to the prior art as it significantly reduces the number of enzyme activity assays at serial dilutions of drug, that had to be made using prior art technology. The measurement of enzyme activity in the absence of drug can also be used for determination of the viral load of the patient, further increasing the amount of information that can be obtained in a single assay run.

It is thus an object of the present invention to provide an improved testing procedure for drug susceptibility integrated with viral load for a virus recovered from an individual.

The present invention thus provides a method for assessing susceptibility of a virus to treatment with a drug inhibiting an enzyme of the wild-type virus comprising the steps:
a) extracting the viral enzyme from a sample containing the virus;
b) measuring the viral enzyme activity both in the absence of the drug and in the presence of the drug at a single predetermined concentration; and
c) determining the virus' susceptibility to treatment with the drug from the relation between the enzyme activities in the presence of the drug and the absence of the drug.

The present invention also provides a method for assessing whether a patient treated for a virus infection is in need of a change in enzyme inhibiting drug therapy due to the virus' resistance to the drug used for treatment, comprising the steps:
a) extracting the viral enzyme from a patient sample containing the virus;
b) measuring the viral enzyme activity both in the absence of the drug and in the presence of the drug at a predetermined concentration; and
c) determining the virus' susceptibility to treatment with the drug from the relation between the enzyme activities in the presence of the drug and in the absence of the drug,
   wherein a susceptibility below a predetermined cut-off level is indicative of a need for a change in drug therapy.

The present invention also provides a method for determining the load of a virus, and said virus' resistance to treatment with a drug inhibiting an enzyme of the wild-type virus in a patient sample, said method comprising the steps of:
a) extracting the enzyme from a sample containing the virus;
b) partitioning the extracted enzyme into at least two, a first and a second, aliquots;
c) measuring the enzyme activity in said first aliquot in the absence of the drug and measuring the enzyme activity in said second aliquot in the presence of the drug at a single predetermined concentration;
d) providing a series of standard values correlating the enzyme activity to viral load;
e) determining the viral load in the sample from the enzyme activity in the absence of the drug, based on the enzyme activity standard values; and
f) determining the viral resistance to treatment with the drug from the relation between the enzyme activities in the presence of the drug and the absence of the drug, respectively.

In accordance herewith, for each recovered patient sample, the activity of enzyme is measured in absence of drug and in presence of a single drug concentration respectively.

The enzyme activity recovered in absence of drug is then recalculated into viral load and the ratio between enzyme activity in presence and absence of drug respectively is determined in order to provide information about the susceptibility to antiviral drugs.

The present invention also provides a system for performing at least steps c)-f) of the method above, said system comprising a computer and an apparatus for determining enzyme activity in a sample, said apparatus being configured to transmit enzyme activity values to said computer, and wherein the computer is configured to receive said enzyme activity values and a series of standard values correlating enzyme activity values to viral load, and is programmed to determine the viral load in the sample from the enzyme activity in the absence of the drug, based on the standard values correlating enzyme activity values to viral load; and to determine the viral resistance to treatment with the drug from the relation between the enzyme activities in the presence of the drug and the absence of the drug.

Thanks to the solution according to the invention, a phenotypic test is achieved that enables for evaluation of the effect of a drug on an enveloped virus, such as a retrovirus, present in the patient sample (e.g. plasma). The test, also referred to as “assay”, provides a procedure to perform combined virus quantification and phenotypic drug resistance testing on viral enzyme that is recovered directly from a patient sample. Viral load and drug resistance are measured in two aliquots originating from the same patient sample. The measurements are preferably performed in parallel, (e.g., simultaneously or essentially simultaneously). Thus, valuable information, both about viral load and the drug sensitivity, can be achieved in a time efficient manner from a single run of one biological sample. This leads to advantages for the patient as it may provide quick and well-founded guidance for medical practitioners in further drug treatment therapy. In other words, the method and system according to the invention provides reliable results in a comparably short time span (achievable within a working day only), providing the possibility for a medical practitioner to make a quick judgement of patient status as well as suitable continuous treatment route, for instance change of drug therapy due to developed resistance of the virus.

According to one aspect of the invention, the virus is an enveloped virus.

According to one aspect of the invention, the virus is a retrovirus and the enzyme is a reverse transcriptase (RT) packed into said retrovirus. Thus, one aspect of the invention is directed to a method of combined testing of viral load and phenotypic drug susceptibility in a virus-infected mammalian individual by testing on an RT enzyme packed into an enveloped virus, recovered from a biological sample, such as blood or plasma, from said individual.

In various embodiments, wherein the retrovirus is a lentivirus, such as HIV, SIV, FIV; a β-retrovirus, such as JSRV or MMTV; a 6-retrovirus, such as BLV, HTLV-1 or HTLV-2; or a γ-retrovirus, such as PERV or MMuLV.

According to one aspect of the invention, the drug is a Non Nucleoside Reverse Transcriptase Inhibitor (NNRTI). A number of NNRTI drugs are known, e.g. nevirapine, efavirenz, rilpivirine, etravirine, delavirdine, lersivirine, GSK 2248761, RDEA806, BILR 355 BS, calanolide A, MK-4965, MK-1439 and MK-6186 (Usach et al., 2013), doravirine and elsulfavirine.

According to one aspect of the invention, the drug is a Nucleoside analog Reverse Transcriptase Inhibitor (NRTI). A number of NRTI drugs are known, e.g. zidovudine (AZT/3T-Azido-2′,3T-dideoxythymidine), didasonine (ddl/2T-3T-dideoxyinosine), zalcitabine (ddC/2′-3′-dideoxycytidine), stavudine (d4T/2′,3′-didehydro-2′,3′-dideoxythymidine), lamivudine (3TC/(−)-L-2′,3′-dideoxy-3′-thiacytidine), abacavir (ABC/[(1S,4R)-4-[2-amino-6-(cyclopropylamino)purin-9-yl]cyclopent-2-en-1-yl]methanol), emtricitabine (FTC/2T-deoxy-5-fluoro-3′thiacytidine), tenofovir disoproxil fumarate (TDF), censavudine (4′-ethynyl-d4T), MK-8591 (EFdA/(4T-ethynyl-2-fluoro-2′-deoxyadenosine), adefovir, telbivudine and entecavir.

According to yet another aspect of the invention, the predetermined drug concentration corresponds to the IC₅₀ value for a reference enzyme with known level of drug resistance.

The predetermined drug concentration may also correspond to 10-50 times the IC₅₀ value for the wild-type enzyme for the drug, such as 15, 20, 25, 30, 35, 40, or 45 times the IC₅₀ value for the wild-type enzyme for the drug. As exemplary embodiments, the wild type reverse transcriptase HxB2 is 50% inhibited at 0.3 μM efavirenz and at 2.7 μM nevirapine, i.e. it has an IC₅₀ for these drugs of 0.3 μM and 2.7 μM, respectively. The predetermined drug concentration may thus be a 10-50 fold increase of these concentrations, e.g. 9 μM and 80 μM, respectively, for a 30 fold increase.

In one aspect of the invention, the predetermined drug concentration corresponds to a clinically relevant cut-off value, e.g. a concentration that gives maximal discrimination between susceptible and resistant virus.

According to yet another aspect of the invention, the patient sample is a blood sample, serum sample, plasma sample, a virus preparation from a cell culture, breast milk, saliva, semen, genital secretions, urine, intraperitoneal fluid or cerebrospinal fluid.

It is also within the scope of the present invention to provide a method for assessing whether a patient treated for a viral infection is in need of a change in drug therapy due to the virus resistance to the drug used for treatment, comprising the method according claim 1, wherein an enzyme activity in the presence of drug at the predetermined drug concentration above a predetermined cut-off value is indicative of a need for a change in drug therapy.

The extraction of the viral enzyme can be achieved in various ways. In one embodiment, extraction comprises isolating the virus particles of a sample before lysing them in order to release the virus enzyme (in case of HIV, reverse transcriptase) for use in further assays.

Parallel, simultaneous determination of viral load and the drug sensitivity profile of the individual from the recovered enzyme are performed by using sensitive enzyme assays, which will later be described in more detail.

The enzyme isolation technique described can be used for any retrovirus, however in the present application the method is exemplified by utilization of plasma derived lentivirus RT for virus quantification and drug resistance testing.

In the following, the invention is exemplified by means of several different retroviruses and the corresponding retrovirus encoded enzyme reverse transcriptase (RT), however it is to be understood that the method and system of the invention may be applicable also for other types of retroviruses containing RT enzyme. The invention is also illustrated by a specific method for measuring enzyme activity.

### BRIEF DESCRIPTION OF DRAWINGS

FIGS. 1A-B show a schematic, stepwise overview of the assay according to one example of the invention;

FIG. 2 shows an overview of the virus isolation step according to one example of the invention;

FIG. 3 shows an overview of the washing step according to one example of the invention;

FIG. 4 shows an overview of the RT extraction step according to one example of the invention;

FIG. 5 shows an overview of the RT reaction step according to one example of the invention;

FIG. 6 shows an overview of the conjugate binding step according to one example of the invention;

FIG. 7 shows an overview of the substrate reaction step according to one example of the invention;

FIG. 8A shows an example of inhibition curves and IC 50 values for wt and mutant controls in the drug resistance assay according to one example of the invention;

FIGS. 8B-C show the inhibition curves of FIG. 8A where the inhibition at EFV concentration of 10 μM is highlighted;

FIG. 9 shows the drug addition step in the drug resistance indicator assay according to one example of the invention;

FIG. 10 shows an example of remaining activity results of the drug resistance indicator assay according to one example of the invention;

FIG. 11 shows an example of an RT reaction plate layout according to the invention;

FIG. 12 shows an example of the relationship between IC50 and remaining RT activity;

FIG. 13 shows an example of the correlation between PhenoSense fold increase and the remaining RT activity from the assay according to the invention; and

FIG. 14 shows the relationship between HIV VL determined with Abbott m2000rt real-time PCR and RTa determined with the assay of the invention.

FIG. 15 is a schematic view of the system according to the invention.

FIG. 16 is an illustration of one embodiment of a part of the system according to the invention.

### DETAILED DESCRIPTION

One way of working the invention will now be described, referring mainly to FIGS. 1A and 1B, presenting an overview of the procedural steps to be carried out. Herein is schematically illustrated the extraction of viral enzyme and following parallel, simultaneous determination of viral load and the drug sensitivity profile of the individual from the recovered enzyme by using sensitive enzyme assays.

FIG. 1A illustrates steps I)-IV), and FIG. 1B steps V)-VII), summarised as follows:
I) HIV purification: providing a biological sample, adding an enzyme inactivating agent for inactivating polymerase activity other than that present in the enveloped virion and subsequently removing the enzyme inactivating agent, enzyme activity blocking antibodies, endogenous enzyme activity inhibitors and antiviral drugs;
II) Reverse transcriptase extraction: lysing the virus particle to release the enzyme, and recovering the concentrated purified viral enzyme in a lysate;
III) RT reaction without and with drug: partition the lysate containing RT from step II) into at least two aliquots, and perform enzymatic polymerization process in the absence and in the presence of a chosen drug at a predetermined concentration in parallel;
IV) Conjugate binding: adding an antibody-enzyme conjugate solution for conjugate binding to the polymerized DNA-strands resulting from step III);
V) Substrate reaction: adding a light-emitting substrate for the conjugate enzyme for quantifying the enzyme activity;
VI) Evaluation: for each of the two aliquots (in the presence and absence of drug respectively), convert the light signal from the substrate to activity of the RT present in the lysate; and
VII) Report: for each patient sample, report viral load (VL Result) and HIV drug resistance (HDR), wherein the value obtained for the aliquot without drug is correlated to the amount of virus in the patient (VL result), and the ratio between the results with and without drug present gives RT remaining activity, a measure of the level of drug susceptibility of the virus having infected the respective patient.

The separate steps will now be further described for improved understanding of the method and system of the invention.

Steps I)-II) correspond to the extraction and isolation of viral enzyme, and may either be performed manually or by a programmable automated workstationt e.g. a Tecan Freedom EVO 150 liquid handling workstation.

An exemplary protocol for isolation of viral reverse transcriptase (RT) from biological samples is now to be described. For illustrative, but not limiting, purposes reference is made to mainly FIGS. 2-4 of the appended drawings in connection to describing the above specified steps I)-II).

An amount of “sample additive” is firstly added to a well (FIG. 2). The purpose of the sample additive is to destroy free host enzymes in the plasma while the enzymes contained within the virions remain intact. In one example, sample additive (e.g. 5,5′-dithiobis-(2-nitrobenzoic acid)) is pipetted into the wells of a deep well microplate. Next, a volume of patient sample (e.g. EDTA plasma from HIV infected individuals) is added. The sample additive and the plasma are mixed, e.g. by pipetting, and incubated at room temperature (18-32° C.). The virions are to be purified from sample additive, enzyme activity blocking antibodies, antiviral drugs and other substances present in the plasma that may interfere with quantification of viral RT. Such purification may be achieved by several separation procedures. The protocol described herein is based on the use of magnetic beads with an immobilized anionic ion exchanger (e.g. Dynabeads® SAX in citrate buffer, schematically illustrated in FIG. 2.

Magnetic beads (Dynabeads® SAX) are carefully suspended and the bead slurry transferred to each well in the deep well plate. The suspension is mixed, e.g. by pipetting, and incubated at room temperature. The virus is now bound to the anionic groups on the magnetic beads (see FIG. 2). The deep well plate is transferred to a magnet rack in order to magnetize for a few minutes. The beads with immobilized virus are now attached to the walls of wells and residual plasma/bead buffer waste can now be aspirated.

The beads are then washed, as illustrated in FIG. 3. Aliquots of bead wash solution are added to each well on the plate. The deep well plate is placed on a shaker, where after it is moved to a magnetic rack and magnetized. The bead wash buffer and waste can now be removed/drained by aspiration. The washing step removes enzyme activity blocking antibodies, antiviral drugs and other substances that may interfere with quantification of viral RT. The beads with immobilized virus is now suspended in essentially pure bead wash solution. This buffer is functional for virus purification, but not adapted to the conditions required for the enzymatic reactions of retroviral RTs. In the following washing, the bead wash buffer is replaced by bead conditioning buffer. The bead conditioning buffer comprises recombinant protein A-G to eliminate remaining RT inhibitory antibody. The beads with immobilized virus are now in an RT reaction compatible buffer.

Extraction of enzymatically active RT is next to be performed, schematically illustrated in FIG. 4. Lysis buffer is added to the wells. In one example, such lysis buffer may correspond to an RT assay compatible buffer including a virus lysing detergent e.g. 1.0% Synperonic A11™.

The deep well plate is then incubated for a few minutes where after it is moved to a magnetic rack and magnetized. The lysis buffer causes the virus envelope to break open so that RT is released into the buffer solution. Following lysis, essentially pure RT in lysis buffer (RT lysate) can be aspirated from each well, said lysed virus envelopes being trapped to the walls of the well by means of the magnetic beads. The recovered RT lysates are essentially free from RT blocking antibodies, antiviral drugs and cellular polymerase activity, and can be characterized and quantified with a sensitive RT activity assay, e.g. the Cavidi ExaVir RT assay, which is disclosed in WO 01/01129.

In step III), the level of enzyme activity (e.g. RT activity) in the recovered lysates is to be determined, called “enzyme reaction step”, or in case of HIV viral enzyme, “RT reaction step”. The RT reaction step may be done for instance by using a modification of the Cavidi RT assay (disclosed in WO 01/01129). In accordance herewith, poly(rA) (prA) covalently bound to the wells of a microtiter plate (e.g. a 96 well plate) serves as template for the incorporation of 5-bromodeoxyuridine 5″-triphosphate (BrdUTP) during the reverse transcription step at 37° C. This is schematically illustrated in FIG. 5.

In steps IV)-V), and as shown in FIGS. 6-7, the amount of bromodeoxyuridine monophosphate (BrdUMP) incorporated into DNA is then detected with an alkaline phosphatase (Ap) conjugated anti-BrdU monoclonal antibody. There are several useful commercially available Ap substrates providing various levels of detection sensitivity (e.g. disodium paranitrophenylphosphate, 4-methylumbelliferyl phosphate and DynaLight⁶). The latter is based on chemo luminescence and is one of the most sensitive Ap activity detections systems currently available.

According to the invention, before the RT reaction step, each RT lysate is partitioned into at least two, a first and a second, aliquots. During the subsequent RT reaction step (described above), enzyme activity in said first aliquot is measured in the absence of any drug and the enzyme activity in said second aliquot is measured in the presence of an antiretroviral drug at a predetermined concentration. Said measurements are performed in parallel, that is simultaneously.

Evaluation, step VI), includes examining the RT activity in absence of drug (viral load) and in presence of drug (drug susceptibility) respectively, and the outcome of such evaluation may be summarised and presented to a user in a report, illustrated in the table of FIG. 1B, step VII). The table of step VII) is to be seen as an example, and presents results of said previous evaluation regarding the viral load and the drug susceptibility of the virus having infected the patient. The table herein reports that Patients 1 and 5 are infected by a virus that shows resistance against the ongoing drug therapy, and a change in treatment may be advisable. Patients 2, 3, 4 and 6 are infected by virus that are susceptible to the drug treatment. All of them have a relatively high viral load and have viruses which are susceptible to drug treatment. If these patients are treated with EFV, the results indicate “bad adherence”, i.e. that the patient does not follow the prescribed dosage instructions.

The evaluation step VI) of the assay according to the invention will now be further described in the following.

The RT reaction procedure for the aliquot without a drug present produces results regarding the “viral load” in a patient: i.e. correlating the activity of the recovered RT in the lysate with the amount of virions per volume plasma. Hereby, the output (step VII)) from such “viral load assay” (VL assay) reflects the concentration of HIV virus in the original patient sample.

The RT reaction procedure for the aliquot with an antiretroviral drug present (i.e. the drug resistance indicator assay) is a complement to the VL assay. The results of the drug resistance indicator assay reflect if the HIV virus recovered from plasma sample is developing resistance to the drug treatment. Several of the antiretroviral drugs work by preventing the virus from replicating, e.g. by means of blocking the activity of the reverse transcriptase. The effect of an anti-retroviral drug on reverse transcription can be measured in the RT reaction step of the viral load assay. As understood from the foregoing, the RT in a lysate originates from the HIV particles in the biological sample. If the HIV in a patient is developing resistance to a drug, the RT in the lysate will then show resistance to that drug in the RT reaction step. Compared to a standard RT reaction without drug, the incorporation of BrdUTP in the presence of drug will be less affected if the RT is resistant, and more affected if the RT is drug susceptible.

To make it possible to quantitate resistance and make comparisons between samples, the level of drug susceptibility is traditionally expressed in a unit called the IC50 value (inhibitory concentration 50). This is the drug concentration, which inhibits virus growth in cell culture or the reaction of an enzyme to 50%.

The IC50 value from a drug susceptibility assay can be used in clinical decision making. An IC50 value that exceeds a certain threshold is an indication that a drug in a patient's antiretroviral drug treatment is not working and a therapy switch should be considered (Pironti et al 2017).

According to prior art, a classic IC50 titration is done by means of a producing an inhibition curve where the remaining RT activity is measured in presence of serial dilutions of a drug.

This is exemplified in FIG. 8A. Recombinant HIV RT enzymes with indicated amino acid substitutions in the HIV 1 BH10 sequence were incubated 3 hours in RT reaction solution without drug and in presence of indicated concentrations of EFV. Remaining activity at each drug concentration was calculated as the quota of RT activity in presence of drug and RT activity in absence of drug times 100 and plotted towards the drug concentration in μM. As illustrated herein, the remaining activity decreases for all tested reverse transcriptase variants at increased drug concentrations, however some of the RT variants are less susceptible and others are more susceptible to EFV. The IC50 values found for the enzymes analyzed are indicated by arrows.

According to the invention, evaluation is done by means of an “indicator assay” in which one single drug concentration is chosen for testing the effect on remaining RT activity, herein also called “single point measurement”. This is exemplified in FIGS. 8B and 8C, which highlights the remaining RT activity for each tested sample at 10 μM EFV concentration. Result from the indicator assay is reported e.g. as remaining activity (Ra) after RT reaction in presence of drug at the predetermined concentration.

Using a single drug concentration provides a number of advantages. It becomes possible to rank the samples of a test in the order from drug sensitive (e.g. wild type RT) to drug resistant (e.g. mutant rRT with known susceptibility). Furthermore, only two assays per sample are needed: one for activity without drug and one for the activity at a single drug concentration. The choice of the fixed, predetermined drug concentration depends on the drug to be tested.

In one embodiment the drug concentration is set to correspond to the IC₅₀ concentration for a reference RT with a known susceptibility, for instance the IC₅₀ value for mutant recombinant RT with a certain susceptibility. The drug concentration and the resistant reference RT could be chosen to represent a clinically important cut off, e.g. where it is obvious that the drug treatment is failing. The sample can then be scored as being at least as resistant as the reference or less resistant. Even if no exact IC₅₀ value will be known for the sample, the results can still be used to support a therapy switch decision.

The system according to the invention is schematically shown in FIG. 15. The system (100) comprises a computer (110) and an apparatus (112) for determining enzyme activity in a sample, said apparatus (112) being configured to transmit enzyme activity values to said computer (110), and wherein the computer is configured to receive said enzyme activity values and a series of standard values correlating enzyme activity values to viral load. The standard values are preferably stored in a database (114) which is accessible to the computer. The computer (110) is programmed to determine the viral load in the sample from the enzyme activity in the absence of the drug, based on the standard values correlating enzyme activity values to viral load; and to determine the virus' resistance to treatment with the drug from the relation between the enzyme activities in the presence of the drug and the absence of the drug.

The apparatus for determining enzyme activity (112) may further comprise an apparatus (116), as shown in FIG. 15B, for automated extraction of viral enzyme from a sample, according to the method aspects of the present invention.

The apparatus for determining enzyme activity and for automated extraction of viral enzyme (112, 116) may be embodied by commercially available laboratory equipment such as automated laboratory robots and work stations, magnetic bead separation racks, reaction plates, plate readers, plate washers. The computer (110) may be any computer that can be programmed to perform the calculations included in the methods according to the invention. The computer preferably comprises output means to communicate the results to the user of the system. Such means include, but are not limited to, displays, printers, and communication lines to other means for presenting or storing the results, such as other computers, databases, etc. Illustrative embodiments of the system according to the invention are provided in the examples below.

FIG. 16 is an illustration of an embodiment of the apparatus for determining enzyme activity and for automated extraction of viral enzyme (112, 116). The illustration shows a setup on a commercially available laboratory workstation (Tecan Trading AG, Switzerland). The workbench comprises a reagent rack (1), sample racks (2), pipette tip racks (3), pipette tip waste collector (4), pipette liquid waste collector (5), a magnet (6), plate heater (7), shaker (8), a storage position for the incubation lid (9), buffer rack (10), incubation lid (11), plate washer (12), luminometer (13), rinse solution container (14), washer liquid waste container (15), and plate wash buffer heater (16).

### EXAMPLES

Materials

Separation beads: Suspension of Dynabeads® SAX, 1 μm paramagnetic polystyrene particles with strongly anionic functional group Life Technologies AS of Ullernchausseen 52, PO Box 114, Smestad, N-0309 Oslo, Norway (ThermoFisher company).

Magnetic bead separation plate Alpaqua Magnum FLX

Automated workstationt: Tecan Freedom EVO 100 or EVO 150 liquid handling workstation

Chemo luminescence AP substrate: Dyna Light® Substrate, Life Technologies AS of Ullernchausseen 52, PO Box 114, Smestad, N-0309 Oslo, Norway (ThermoFisher company)

Luminometric plate reader LuMate® Microplate Luminometer, Awareness Technology, Inc

Hydroflex plate washer. Micro plate washer available from Techan.

RT reaction plate: Microtiter plates with immobilised prA, i.e. Nunc™ NucleoLink™ strips (ThermoFisher) coated with prA.(

Cysteine modifying agent: e.g. 66 mM 5,5′-dithiobis-(2-nitrobenzoic acid),

Mild sulfhydryl reducing agent: 33 mM cysteamine in water

Protein A/G: Recombinant protein A/G fusion protein that combines IgG binding domains of both Protein A and Protein G, ProSpec-Tany Technogene Ltd. Rehovot Branch 179 Herzel St. Rehovot 76110, Israel

Antiviral drugs: Nevirapine (11-cyclopropyl-5,11-dihydro-4-methyl-6H-dipyrido[3,2-b:2′,3′-f][1,4]diazepin-6-one) (NVP), Efavirenz, (−)6-chloro-4-cyclopropylethynyl-4-trifluoromethyl-I,4-dihydro-2H-3,1-benzoxazin-2-one) (EFV), Etravirin (4-({6-amino-5-bromo-2-[(4-cyanophenyl)amino]pyrimidin-4-yl}oxy)-3,5-dimethylbenzonitrile) (ETV) and Rilpivirin (4-{[4-({4-[(1E)-2-cyanoeth-1-en-1-yl]-2,6-dimethylphenyl}amino)pyrimidin-2-yl]amino}benzonitrile) (RPV) were purchased from Sequoia Research Products Ltd, United Kingdom.

3′-azido-3′-deoxythymidine triphosphate (AZT-TP) was bought from Moravek Biochemicals, California US.

Plasma samples from HIV infected individuals: Plasma samples from HIV infected South African blood donors were purchased from South African National Blood Service (SANBS), Biorepository, Hospital Road, Boksburg).

Recombinant RT enzymes: RTs with NNRTI specific mutations. Recombinant RT with C-terminal His-tag_pET-30a(+) was constructed from the HIV BH10 isolate used as WT RT sequence, introducing mutations to specific positions. The plasmid construct was purchased from Genescript. Each rRT was expressed from a plasmid with the desired sequence, not by site directed mutagenesis of the WT BH10 sequence. Plasmid DNA constructs with HIV-RT wt, L100I, K103N/L100I and Y181C were transformed into BL21 (DE3). The RT proteins were purified on a 4 ml Ni2+-sepharose column.

The RTs with AZT specific mutations were produced by introducing the mutations into the RT-coding region of a wild type HXB2-D EcoRl-Ndel restriction enzyme fragment cloned into the expression vector pKK233-2 (Amersham Biotech). Mutations were generated using QuikChange (Stratagene). The mutated vectors were transformed into E. coli strain XL1-Blue and the genotypes were verified by DNA sequence analysis.

The recombinantly expressed and purified SIV RT was derived from the molecular clone pSIVₛₘ/H4. An expression vector pSRT.ET 11c was constructed and expression of SIV-RT in E. coli strain BL21 (DE3) in ordinary LB medium was carried out. The enzyme was purified in three chromatographic steps on a Heparin Sepharose CL-6B column, a Sepharose FF column and a Phenyl Sepharose CL-4B column.

The Mouse mammary tumor virus (MMTV) recombinant RT was a gift from A Hizi Tel Aviv University. The MMTV RT gene was derived from the pUC1002 provirus plasmid generated from the MMTV BR6 strain. The RT was expressed in E. coli DH5a and purified from bacterial extracts. (Taube et al., 1998).

Recombinant MMuLV RT, prepared according to Roth et al. (1985), was purchased from Pharmacia (catalogue no. 27-0925).

Virus preparations: FIV virus FIV-M2 clade B containing cell culture supernatant, cleared from cell debris by centrifugation, was obtained courtesy of Dr Donatella Matteucci, Retrovirus Centre and Virology Section, Dept. of Biomedicine, University of Pisa, Italy (Matteucci et al, 1995). The supernatant obtained was aliquoted and stored at −70° C. until use.

Human T-cell lymphotropic virus type 1 and type 2 of cell culture origin was a gift from Dr Bo Svennerholm, Sahlgrenska hospital, Goteborg, Sweden.

Bovine leukemia virus (BLV) orginating from FLK-BLV cells was obtained by the courtesy of Dr Malik Merza, Svanova AB, Uppsala Sweden. (This cell line is chronically infected by BLV).

Virus culture supernatant was cleared from cell debris by centrifugation, aliquoted and stored at −70° C. until use

The UK strain of Jaagsiekte Sheep Retrovirus (JSRV), was obtained courtesy of Massimo Palmarini, Moredun Research Institute, 408 Gilmerton Road, Edinburgh EH17 7JH, UK. The supernatant obtained was aliquoted and stored at −70° C. until use.

PERV from PK15 cells were obtained by the courtesy of Dr Jonas Blomberg, Section of Clinical Microbiology, Department of Medical Sciences, Uppsala University, Sweden. Virus culture supernatant was cleared from cell debris by centrifugation, aliquoted and stored at −70° C. until use

Buffers used:

Bead wash buffer: 0.2 M citrate/citric acid, 0.2 M NaCl, 0.1% Triton X305, pH 6.0.

Bead Reconditioning Buffer: An RT assay compatible buffer e.g. 10 mM Hepes (N-(2-Hydroxyethylpiperazine-N′-(2-ethanesulfonic acid), 6.25 mM KAc, 50 mM MgCl2*6H2O, 0.175 mM EGTA, 9.75 ng odT22, 2.0 mM Spermine, 0.3% Triton CF32, 10 μg/ml Protein A/G, 0.5 g/L BSA pH 7.4 mM.

Lysis buffer: An RT assay compatible buffer including a virus lysing detergent e.g. 1.0% Synperonic A11, and identical concentrations of the same components as in the conditioning buffer. A sulfhydryl reducing agent, i.e. 2 mM cysteamine is optionally added when processing viruses with RT that are sensitive to SH oxidation/modification.

RT Reaction solution: e.g. 10 mM Hepes pH 7.6, 19 μM BrdUTP, 80 ng/ml odT₂₂, 4 mM MgCl₂, 2 mM spermine, Synperonic A11 0.5% (v/v), EGTA 0.2 mM and BSA 0.5 mg/ml, GTP.

Drug reaction solution: A buffer identical to above mentioned RT Reaction solution with a defined concentration of an antiviral drug added.

NRTI RT Reaction buffer was supplemented with 6 mM ATP, the pH was adjusted to 7.0 and the final BrdUTP concentration was decreased to 1.5 μM.

NRTI drug RT reaction buffer was supplemented with 0.70 μM AZT-TP.

All buffers used for analysis of γ-retroviruses RTs were supplemented with 6 mM Mn2+, but were otherwise identical to the buffers described above.

Plate wash buffer: Boric acid 3 mM, 0.75% Triton X-100, 0.005% (W/V) Dextran Sulphate, 0.2% ETOH and 0.025% NaN₃.

Virus Abbreviations
HIV Human immunodeficiency virus
SIV Simian immunodeficiency virus
FIV Feline immunodeficiency virus
JSRV Jaagsiekte Sheep Retrovirus
MMTV Mouse Mammary Tumor Virus
BLV Bovine Leukemia Virus
HTLV Human T-lymphotropic virus)
PERV Porcine endogenous retrovirus
MMuLV Moloney Murine leukemia virus

### Example 1: Viral Load Assay

It is a purpose of this example to illustrate one way of performing a viral load assay according to the invention.

Frozen plasma samples from HIV infected individuals were thawed and mixed.

Isolation of HIV is performed in a separation plate, a 96 deep-well microplate (well volume 2 ml). The Viral Load assay is started by addition of 40 μl sample additive e.g 5,5′-dithiobis-(2-nitrobenzoic acid) to each well (see FIG. 2). Thereafter the patient plasmas and control samples are added. Sample additive is mixed with the plasma prior to isolation of HIV for inactivation of certain enzymes present in the plasma. After 20 minutes incubation, a slurry containing 1 μm magnetic polymeric beads, is added to the wells. The positively charged beads are used to capture the HIV particles. At low pH (e.g. 5.5) and appropriate salt concentration the negatively charged HIV particles bind to the beads while most of the other components present in plasma will not bind, e.g. the proteins (antibodies) and anti-retroviral drugs (ARVs). After an additional five minutes incubation, the microwell plate is transported to a magnetic plate. When exposed to the magnetic field from the magnetic plate, the beads will be trapped to the walls of the wells. The plasma can then be removed by aspiration using a pipette. The separation plate is then transported back to the shaker, away from the magnetic plate.

The wash procedure (see FIG. 3) starts by adding a first wash buffer, the Bead Wash Buffer, and the entire separation plate will be shaken during three minutes to resuspend the beads. After that, the separation plate is moved to the magnetic plate where the beads will be exposed to the magnetic field and trapped to the walls of the wells. Next, the wash buffer is aspirated. The separation plate is then transported back to the shaker, away from the magnetic plate. In the current example, this wash procedure is repeated three times with the Bead Wash Buffer (referred to as “Wash solution 1” in FIG. 3) and one time with the second wash buffer, the so called Bead Reconditioning Buffer (referred to as “Wash solution 2” in FIG. 3).

The composition of the Bead Wash Buffer is similar to the binding buffer (pH 5.5, salt) but also contains a non-lysing detergent to increase the wash efficiency. Accurate wash temperature is advantageous for the result, so therefore the Bead wash buffer temperature is preferably controlled. The Bead Reconditioning Buffer has higher pH (e.g. 7.0) and also contains Protein A/G to remove unwanted plasma antibodies. After the wash procedure the HIV particles still bind to the beads but very few contaminants from the plasma remain.

The reverse transcriptase (RT) is now to be extracted from the viral particles, which is illustrated in FIG. 4. The HIV particle is composed of a protein core surrounded by a lipid bilayer envelope that will break if it is exposed to certain detergents. Once the lipid bilayer envelope is dissolved, the protein core falls apart and the RT inside is released. When the last wash step is finalized, lysis buffer containing e. g. the detergent Synperonic A11 is added to the wells, in FIG. 4 referred to as “lysis buffer”. The virus envelope breaks and the reverse transcriptase, RT, is released. With the magnetic beads still bound to the walls of the wells, the lysate containing the released RT is collected by aspiration and used in the subsequent RT reaction step.

RT Reaction

Referring now to FIGS. 5-7, the RT reaction is the enzymatic polymerization process whereby the enzyme RT builds a new single-stranded DNA strand. To do this it needs an RNA template to copy, a short complementary DNA strand to start extending from and a complementary deoxynucleotide(s) triphosphates that can be used as building blocks in the growing strand. The reaction is performed in an RT reaction plate, in this example a 96-well plate (well volume 300 μl). The well surfaces are pre-coated with prA, i.e. a single stranded polyriboadenylic acid composed of only one nucleotide base, adenine. The prA molecules have an average chain length of >200 nucleotides and act as the template for the RT reaction.

The other components needed for the RT reaction are part of the RT reaction mixture. The RT reaction mixture (see FIG. 5) contains odT₂₂ (oligo-deoxy-thymidine-22-mono-phosphate) and BrdUTP (Bromo-deoxy-Uridine-tri-phosphate). odT₂₂ is the primer, and corresponds to a short (22 bases) single stranded deoxyribonucleic acid oligonucleotide base pairing with the prA strand. BrdUTP provides the nucleotide bases that get added to the primer by the RT. Directly after the addition of the RT reaction mixture, also the lysate containing the RT enzyme is transferred to the respective well.

In this step an HIV-1 rRT calibrator is introduced in the assay. The calibrator has already been prepared in parallel with the earlier described separation/washing process and is based on a recombinant HIV-1 reverse transcriptase with known activity. The lyophilized, (freeze dried) calibrator has been dissolved, mixed and serially diluted into a set of concentration levels, herein a number of six levels. These are added into separate wells in the RT reaction plate and will be used to construct a calibration curve.

With the addition of lysate or calibrator, the RT-enzyme reaction is now started and the amount of the newly produced BrdU strand will be proportional to the amount of RT enzyme present in the lysate or calibrator.

The RT reaction is temperature dependent and reaction velocity increases with increasing temperature until enzyme function is impaired by denaturation. The RT reaction plate is therefore placed on a heating block during the reaction and also covered by a lid to prevent evaporation during the 3 hour incubation. Both the incubation temperature and the time for the reaction (incubation time) are factors that are to be controlled.

The RT reaction is stopped after 3 hours by removing the RT-reaction solution. This is done by a careful wash procedure using the Hydroflex plate washer. The wash solution used contains certain components e.g. detergent to increase the efficiency of the wash.

Conjugate Binding

After the RT-reaction solution is washed away, an antibody-enzyme conjugate solution is added (see FIG. 6). The antibody part of the conjugate is a monoclonal mouse antibody with anti-BrdU specificity and the conjugate enzyme is alkaline phosphatase (AP). The conjugate binding reaction is also temperature dependent and the RT reaction plate is therefore placed back on the heating block during the 37° C., 30 min incubation.

The conjugate binding step is ended by a careful wash procedure using the Hydroflex plate washer to remove the conjugate solution and non-specifically bound conjugate so that only conjugate bound to the BrdU remains. Increased wash temperature (30-35° C.) is advantageous for a good wash result. The wash solution is the same as for the RT wash.

Substrate Reaction

In a final reaction, a solution containing a substrate for alkaline phosphatase (AP), is added to the wells (see FIG. 7). This substrate generates light when the AP enzyme part of the conjugate modifies the substrate structure. In this stage of the process the chemical reaction can be affected by airborne dust particles that may contain AP. Therefore the RT reaction plate is directly moved into the Lumate Microplate Luminometer reading chamber where it is protected from dust. After a short “lag phase” of 10 minutes to obtain a stable light signal, the light intensity is measured by the luminometer.

Evaluation

The purpose of the evaluation is to convert the light signals from the RT assay to the desired output; i.e. the HIV viral load in the plasma sample and thus in the infected patient. The intensity of the light produced by the AP enzymatic process is proportional to the amount of conjugated enzyme bound to the well via the binding of the anti-BrdU antibody part to the synthesized BrdU strands. The amount of incorporated BrdU is in its turn proportional to the amount of RT that was present in a lysate or in a calibrator respectively. The HIV rRT calibrators are used to construct a calibration curve which is used to quantify the amount of RT activity in the lysates. The primary output according to the formula for the regression line from the calibration curve is RTa units/ml plasma. One RTa unit corresponds to the activity generated by 1 fg HIV-1 BH10 calibrator enzyme. This output from the RT assay reflects the concentration of HIV virus in the starting human plasma sample. The standard unit to express HIV concentration is RNA copies/ml plasma.

The concentration of RT in the lysate will be converted to the concentration of HIV RNA in the sample by using a conversion factor. This conversion factor is empirically determined by correlating the results obtained with a state of the art method (e.g. Roche Cobas Amplicor assay), expressed as HIV RNA copies, with the results from the same plasma cohort run on the assay.

With the viral load assay, the response to an antiretroviral drug treatment is reflected by alterations in amount of circulating virus.

### Example 2: Drug Resistance Indicator Assay

This example presents a setup for a semi-quantitative drug resistance indicator assay. Each sample is tested against a single predetermined drug concentration (instead of against several concentrations as in a standard IC₅₀ drug resistance assay).

The drug resistance indicator assay procedure follows that of the Viral Load procedure, but the RT assay step and the Evaluation are performed differently. As illustrated in FIG. 9, in the RT reaction step either buffer with the selected drug at a single concentration or buffer without drug is added to the wells with the RT reaction mixture before the lysate is added.

In this example, the drug used in the test was the non-nucleoside reverse-transcriptase inhibitor (NNRTI) Efavirenz, and the predetermined concentration was 10 μM, corresponding to the IC₅₀ value of mutated HIV rRT L100I, chosen to represent the resistant reference (see FIG. 10). HIV rRT L100I is a well characterized HIV sample representing a virus with known drug resistance mutation. Thus, when RT reaction step is evaluated, the remaining activity for L100I should be close to 50% as the drug concentration used is the known IC₅₀ value for this reference enzyme.

Each lysate is split into two aliquots, one whereof is added to a well containing drug at a predetermined concentration, and one whereof is added to a well without added drug. Apart from the lysates, said HIV drug susceptibility reference samples are also included in the assay. The lyophilized, (freeze dried) reference has been dissolved and mixed and is then added into wells with and without drug in the RT reaction plate just like the lysates.

During the RT reaction, the drug present in the assay will be more or less effective in blocking the RT in the different lysates. A lysate with drug-susceptible RT will be blocked and very little new DNA will be made. In a lysate with a drug-resistant RT similar to the reference, the drug will block about 50% of the BrdUTP incorporation. In a lysate with a drug resistantant RT more resistant than the reference, the drug may not block incorporation of BrdUTP at all.

Evaluation Drug Resistance Indicator Assay

The drug resistance indicator assay gives at least two different answers:
I. The Viral Load value indicating whether the ongoing therapy is effective; and
II. The Drug Susceptibility of the virus replicating in the patient and whether an elevated viral load recorded is due to the patient's adherence to the treatment of the patient or occurrence of drug resistant virus.

The light signal corresponding to the RT activity in wells without drug added is set to 100%. The signals obtained from the wells with drug are calculated as a percentage of the activity found without added drug. An example of presentation of remaining activity is shown in FIG. 10. Herein is seen that the remaining activity of the resistant reference is close to 50%. This is expected as the drug concentration used is the known IC₅₀ value for this rRT. This result indicates that the assay has been run under proper conditions and that the assay run can be accepted. The remaining activities for the samples 1-8 are calculated in the same manner.

With cut-off values established by using existing drug resistance databases and/or by clinical studies the remaining activity relative to a known resistant reference RT can be interpreted as infection with susceptible, reduced susceptible or resistant virus.

### Example 3—Viral Load and Drug Resistance Assay

This Example aims at describing one possible outline for the combined virus quantification and phenotypic drug resistance testing on viral RT enzyme that is recovered directly from patient plasma samples. The example presents a protocol for testing 28 patient samples for viral load and drug susceptibility, and also the evaluation and resulting report.

FIG. 11 illustrates an example of an RT reaction plate layout for measuring the activity of the purified RT containing lysates in presence and absence of a drug at a carefully selected/predetermined concentration. For each sample, HIV particles were isolated and viral RT extracted using the method previously described (see e.g. Example 1).

In this example, the drug chosen for the assay is Efavirenz, and the predetermined concentration is set to 10 μM, corresponding to the IC₅₀ value for mutated recombinant control RT L100I, in respect of the chosen drug. FIG. 11 is understood to represent an example only, and the skilled person can think of a wide variety of alternative, equally possible RT reaction plate layouts. For instance, it is possible to choose between a wide variety of control RTs and corresponding concentration (based on IC₅₀ value), different number of drug controls etc.

In the table of FIG. 11, the following abbreviations are used:
DRS=Drug Reaction Solution
VLRS=RT Reaction Solution
Pos control=Positive control
Neg control=Negative control
wt rRT=HIV-1 wild type recombinant Reverse Transcriptase
bg=background
1^{st}-9^{th} std=1^{st}-9^{th} serial diluted HIV-1 rRT calibrator

Moreover, “Drug reaction solution” is understood to refer to a reaction buffer with a selected antiretroviral drug dissolved to a predetermined concentration, and “RT reaction solution” is understood to refer to a reaction buffer without any drug present. For examples of buffers, see “Buffers used” under “Materials”.

In this protocol, 28 patient samples are tested on the same microplate, each sample being evaluated for viral load and drug susceptibility respectively, using the drug resistance indicator assay described in Example 2 for evaluating viral drug susceptibility.

Referring to the table of FIG. 11, schematically illustrating the outline of a microplate, the patient sample wells correspond to A1-D7, A8, B8, C8 and D8. Wells E7, E8, F7 and F8 correspond to positive controls (a non-infectious recombinant HIV virus preparation used as separation control); wells G7, G8, H7 and H8 correspond to negative controls (plasma from healthy blood donors); A9, A10, B9 and B10 correspond to control wells to be loaded with resistant recombinant RT with defined drug susceptibility (herein SIV rRT); C9, C10, D9, D10, E9, E10, F9 and F10 correspond to control wells to be loaded with intermediate recombinant RT (herein L100I rRT and Y181C rRT respectively); G9, G10, H9 and H10 correspond to control wells to be loaded with wild-type recombinant RT; A11-H11 and A12 correspond to serial dilutions of a recombinant control RTs with defined enzymatic activity; and B12-H12 correspond to background control wells.

In the given example, SIV rRT, L100I rRT, Y181C rRT and wt rRT are recombinant control RTs with defined drug susceptibility.

Loading of the wells on the RT reaction plate is now to be described.
Add 30 μl Drug reaction solution to all wells in columns 1, 3, 5, 7 and 9 at the 96 wells RT reaction plate.
Add 30 μl RT reaction solution to all wells in columns 2, 4, 6, 8, 10, 11 and 12 at the RT reaction plate.
Add 100 μl Lysis buffer to wells B12-H12 at the reaction plate (background controls).
Add 100 μl serial dilutions of a recombinant RT standard to wells A11-H11 and A12.
Add 100 μl of recombinant HIV RT control 1 (herein SIV rRT) with defined susceptibility to the selected drug to wells A9 to A10, and B9 to B10 respectively.
Add 100 μl of recombinant HIV RT control 2 (herein L100I) with defined susceptibility to the selected drug to wells C9 to C10 and D9 to D10.
Add 100 μl of recombinant HIV RT control 3 (herein Y181C) with defined susceptibility to the selected drug to wells E9 to E10 and F9 to F10.
Add 100 μl of wt HIV-1 RT to wells G9 to G10 and H9 to H10.
Add 100 μl of sample lysates from the separation plate to wells A1-H8. Each lysate is analysed as duplicates in two adjacent wells; one sample in presence of drug and one in standard reaction solution without drug (see plate layout in FIG. 11).
Incubate the RT reaction plate for 3 h at 37° C.
The RT reactions are finally terminated by repeated washes of the RT reaction plate and the amount of newly synthetized DNA in each well are determined using an AP conjugated anti-BrdU monoclonal antibody as described previously. A chemoluminiscent AP substrate (DynaLight® Substrate) is used to achieve high detection sensitivity in the final AP assay.

Calculation of VL and Drug susceptibility.

The signals generated by the DynaLight® Substrate in the AP reaction are read in a luminometric plate reader. The serially diluted rRT calibrator provide a series of values correlating the enzyme activity to viral load. The signals obtained are plotted against the amounts of rRT used and the formula for the standard regression line is calculated. This formula is then used to recalculate the signals generated from the lysates measured in absence of drug into amount of RT/ml lysate and, after consideration of lysis recovery and samples volumes, into amount of RTa units/ml original plasma.

The virus susceptibility to treatment with the drug can be determined from the relation between the enzyme activities in the presence and in absence of the drug. This can be expressed as percent remaining RT activity, i.e. the quota between signal in presence of drug and signal in absence of drug times 100.

### Example 4—Determination of Susceptibility to Four NNRTI Drugs

Table 1 demonstrates of the ability of the current invention to determine susceptibility to four currently used NNRTI drugs. The plasma samples Sa 24 and Sa 147 were processed according to the above described protocol for extracting viral RT. The RT extracts recovered from this process and the other RT samples were analyzed for remaining activity according to above described drug resistance indicator assay. The drug concentrations used were Efavirenz (EFV) 9.0 μM, Nevirapin (NPV) 81.0 μM, Etravirin (ETV) 21.8 μM and Rilpivirin (RPV) 12.0 μM.

TABLE 1

| | | | Remaining activity (% of actvity in absence of drug) | | | |
|---|---|---|---|---|---|---|
| Virus | Sample type | Sequence/Isolate | EFV | NPV | ETV | RPV |
| HIV-1 B | Recombinant RT | HXB2 wild type | 4.1 | 8.1 | 2.8 | 2.4 |
| HIV-1 B | Recombinant RT | BH10 wild type | 3.5 | 3.4 | 3.5 | 2.5 |
| HIV-1 B | Recombinant RT | HXB2 T215Y | 3.4 | 8.3 | 1.5 | 1.6 |
| HIV-1 C | Plasma Sa72 | wild type | 8.3 | 16.1 | 6.5 | 9.3 |
| HIV-1 B | Recombinant RT | BH10 V179D | 19.1 | 5.1 | 2.2 | 3.0 |
| HIV-1 B | Recombinant RT | BH10 Y181C | 17.3 | 72.6 | 38.7 | 20.8 |
| HIV-1 B | Recombinant RT | BH10 L100I | 63.8 | 30.7 | 28.4 | 46.6 |
| HIV-1 B | Recombinant RT | BH10 K103N | 60.8 | 64.1 | 5.2 | 13.9 |
| HIV-1 C | Plasma Sa24 | K103N | 78.0 | 69.3 | 24.2 | 31.8 |
| HIV-1 B | Recombinant RT | BH10 K103N/L100I | 96.3 | 72.5 | 47.3 | 82.3 |
| HIV-1 C | Plasma Sa147 | K103N, E138Q, Y318F, M184V | 87.8 | 94.9 | 68.0 | 80.8 |
| SIV | Recombinant RT | SIVsmH4 | 99.9 | 99.5 | 98.5 | 107.5 |
| FIV | Cell culture virus | M2 CladB | 93.6 | 87.7 | 90.6 | 86.1 |

The remaining activity profile pattern for the four drugs differed between the samples.

The three wild type RTs and the RT with the NRTI induced mutation T215Y RT were susceptible to inhibition by all four drugs. The samples containing one or two known NNRTI mutations revealed varying degree of reduced susceptibly or resistance to the different drugs. Simian immune deficiency virus (SIV) RT, Feline immune deficiency virus (FIV) RT and RT from Sa 147, a plasma sample with a HIV with four NNRTI specific mutations in the RT gene, were resistant to all four drugs.

### Example 5—Determination of Susceptibility to Both NRTI and NNRTI Drugs

TABLE 2

| | | | Remaining activity (%) | |
|---|---|---|---|---|
| Virus | Sample type | Sequence/Orgin | AZT | EFV |
| Lenti virus | | | | |
| HIV-1 B | Recombinant RT | HXB2 wild type | 1.8 | 4.1 |
| HIV-1 B | Recombinant RT | HXB2 T215Y | 11.6 | 3.8 |
| 69SS + 5M | Recombinant RT | HXB2 M41L,T69S-SS, L210W, | 52.6 | 3.5 |
| | | R211K, L214F, T215Y | | |
| 69SG + 5M | Recombinant RT | HXB2 M41L,T69S-SG, L210W, | 66.3 | 3.9 |
| | | R211K, L214F, T215Y | | |
| HIV-1 B | Recombinant RT | BH10 wild type | 2.9 | 2.4 |
| HIV-1 B | Recombinant RT | BH10 V179D | 6.2 | 19.1 |
| HIV-1 B | Recombinant RT | BH10 Y181C | 4.2 | 15.8 |
| HIV-1 B | Recombinant RT | BH10 L100I | 6.4 | 63.8 |
| HIV-1 B | Recombinant RT | BH10 K103N | 6.0 | 60.8 |
| HIV-1 B | Recombinant RT | BH10 K103N/L100I | 10.7 | 96.3 |
| HIV-2 | Cell culture virus | CRF01_AB | 4.3 | 100.2 |
| SIV | Recombinant RT | SIVsmH4 | 19.0 | 103.0 |
| FIV | Cell culture virus | M2 CladB | 15.4 | 93.6 |
| β -retrovirus | | | | |
| JSRV | Cell culture virus | UK strain | 8.6 | 102.7 |
| MMTV | Recombinant RT | BR6 strain | 7.7 | 101.6 |
| δ-retrovirus | | | | |
| BLV | Cell culture virus | FLK-BLV cells | 6.9 | 101.7 |
| HTLV-1 | Cell culture virus | MT-2 Cells | 6.6 | 93.8 |
| HTLV-2 | Cell culture virus | | 4.1 | 91.7 |
| γ -retrovirus | | | | |
| PERV | Cell culture virus | PERV-PK15 | 2.7 | 98.0 |
| MMuLV | Recombinant RT | Roth et al. (1985) | 2.2 | 96.9 |
| Mammalian* | | | | |
| γ -polymerase | mithochondria | Beef Heart | 100.5 | 94.8 |

*The mammalian γ- polymerase has an editing function and is not affected by AZT-TP

Table 2 demonstrates of the ability of the current invention to determine susceptibility to both NRTI and NNRTI drugs. The RT preparations investigated were analyzed for remaining activity according to the protocol for drug resistance indicator assay. Note that two different RT reaction conditions were used. Susceptibility to NNRTIs was determined at saturated deoxynucleotide substrate concentrations, while the reaction conditions for NRTI susceptibility assay was designed as a compromise between the conditions required for a rapid DNA elongation reaction and ability to distinguish between resistant and susceptible RTs (see RT reaction buffers in material and methods). The drug concentrations used were AZT-TP 0.16 μM and (EFV) 9.0 μM respectively.

In presence of 0.16 μM AZT-TP the two HIV-1 recombinant wild type RTs investigated exhibited remaining activities (Ra) of 1.8 and 2.9% respectively. The Ra value increased to 9.4 when the mutation T215Y was introduced in the sequence of HIV-1 HXB2. RTs containing multiple NRTI associated mutations like M41L, T69S-SS, L210W, R211K, L214F, T215Y exhibited Ra values in the range 53-66%. None of these mutations did significantly affect the susceptibility to NNRTI drugs like EFV. These data show that the current invention also can be used to characterize susceptibility to chain-terminating NRTI drugs like AZT.

All enzymes in the panel of HIV-1 RTs with NNRTI specific mutations exhibited similar susceptibility to AZT-TP inhibition. This type of mutations did thus not influence the susceptibility to AZT-TP. Wild type RTs from other lentiviruses like HIV-2, SIV and FIV had similar or slightly higher AZT Ra values as the wild type HIV-1 RTs.

RTs from β-retroviruses like Jaagsiekte Sheep Retrovirus (JSRV) and Mouse Mammary Tumor Virus (MMTV) were susceptible to AZT-TP inhibition but showed slightly higher Ra values (7.7-8.6%) than the wild type HIV-1 RTs.

RTs from 6-retroviruses like Human T-cell lymphotropic virus type 1 (HTLV-1) and Bovine leukemia virus (BLV) gave similar results as those from β-retrovirus.

RTs from γ-retroviruses had Ra values in the same range as the wild type HIV RTs

Among all the analysed enzymes only the HIV-1 RTs devoid of major NNRTI specific mutations were significantly inhibited by EFV.

The mammalian γ-polymerase, that was included as a control was not significantly inhibited by any of the anti viral drugs.

The drug resistance indicator assay thus has the capacity to analyse polymerase samples from viruses and cells for susceptibility to both NRTI and NNRTI drugs.

### Example 6—Relationship Between IC50 and Remaining RT Activity

The example aims at showing that remaining RT activity at a single selected drug concentration gives virtually identical information as a IC50 titration based on multiple drug concentrations

The IC50 value for EFV was determined for each preparation in a panel of 10 recombinant HIV RT enzymes with defined amino acid substitutions in the HIV 1 BH10 sequence. The duration of the RT reactions were 3 hours and each IC50 value was calculated based on measurements of the RT activity at eight different EFV concentrations and in absence of drug (as exemplified in FIG. 8A).

The Ra value for each enzyme was calculated from the same data set using the RT activity at 9 μM EFV divided with the RT activity in absence of drug times 100 (as exemplified in FIGS. 8B and C).

FIG. 12 shows the Ra value, at the selected single EFV concentration 9 μM for each enzyme, plotted against the IC50 value of the same enzyme. Each dot in FIG. 12 thus represents one RT preparation.

It is clear from FIG. 12 that there is a strong correlation between the remaining activity at 9 μM EFV and the corresponding IC50 value (r2=0.9859, p<<0.001).

### Example 7—Correlation to Phenotypic Data

This Example aims at showing that the method for evaluating RT activity according to the present invention gives information that is comparable to that provided by the well-established PhenoSense™ test. PhenoSense is a widely used in vivo phenotypic test for HIV drug resistance that reports the fold increase of the samples IC50 relative to the IC50 value for a reference drug-sensitive HIV clone. It can be regarded as gold standard drug resistance assay.

A panel of recombinant HIV-1 RTs constructed by introduction of NNRTI specific mutations into the sequence of HIV-1 BH10 wild type RT were investigated using the current protocol for drug indicator assay according to the invention. The duration of the RT reactions were 3 hours and the samples were analyzed in presence and absence of 9 μM EFV.

The Stanford HIV Drug Resistance database contains a table with RT drug resistance mutations and corresponding PhenoSense fold increase. Some of the RTs with known mutations that had been tested with the drug resistance indicator assay in examples 3 to 5 could be found in this table. The relationship between remaining RT activity and the phenotypic fold increase can thus be analyzed. As can be extracted from FIG. 13 we found a strong correlation (r2=0.9738, p<0.001) between PhenoSense fold increase and the remaining RT activity from the current drug resistance indicator assay.

### Example 8—Relationship Between HIV VL Determined with Abbott m2000rt Real-Time PCR and RTa Determined with the Current Invention

This Example aims at showing that the method for evaluating RT activity according to the present invention is comparable to the standard method of evaluating viral presence based on RNA content detected by means of rtPCR.

CPD plasma samples from 47 HIV infected persons from South Africa were analyzed using the viral load assay according to the invention (described in example 1). The serially diluted standard with a known concentration of recombinant HIV RT is used to recalculate the signals generated from the sample RTs measured in absence of drug into amount of RT/ml lysate and, after consideration of lysis recovery and samples volumes, into amount of RTa units/ml original plasma. The plasma samples had previously been analyzed for HIV-1 RNA viral load with Abbott m2000rt real-time PCR. FIG. 14 shows the amount of RT recovered (RTa units) plotted against the amount of HIV-1 RNA (RNA copies) for each of these 47 plasma samples. As can be extracted from the figure, there is a strong correlation (R²=0.8873, p<0.001) between these variables.

### REFERENCES

Bryant L, Smith N, Keiser P. A Model for Reduced HIV-1 Viral Load Monitoring in Resource-Limited Settings. J Int Assoc Provid AIDS Care 2013 12: 67-71.
Goodall R L, Dunn D T, Pattery T, et al. DART Virology Group and Trial Teams. Phenotypic and genotypic analyses to guide selection of reverse transcriptase inhibitors in second-line HIV therapy following extended virological failure in Uganda. J Antimicrob Chemother 2014; 69:1938-44.
Gupta S, Palchaudhuri R, Neogi U, Srinivasa H, Ashorn P, De Costa A, Källander C, Shet A. Can HIV reverse transcriptase activity assay be a low-cost alternative for viral load monitoring in resource limited settings? BMJ Open 2016; 6:e008795. doi:10.1136/bmjopen-2015-008795
Hamers R L, Sigaloff K C, Wensing A M, et al. PharmAccess African Studies to Evaluate Resistance (PASER). Patterns of HIV-1 drug resistance after first-line antiretroviral therapy (ART) failure in 6 sub-Saharan African countries: implications for second-line ART strategies. Clin Infect Dis 2012; 54:1660-9.
Hamers R L, Schuurman R, Sigaloff K C, et al.; PharmAccess African Studies to Evaluate Resistance (PASER) Investigators. Effect of pretreatment HIV-1 drug resistance on immunological, virological, and drug-resistance outcomes of first-line antiretroviral treatment in sub-Saharan Africa: a multicentre cohort study. Lancet Infect Dis 2012; 12:307-17.
Huang D, Zhuang Y, Zhai S, Song Y, Liu Q, et al. HIV reverse transcriptase activity assay: a feasible surrogate for HIV viral load measurement in China. Diagn Microbiol Infect Dis 2010 68: 208-213.
Labbett W, Garcia-Diaz A, Fox Z, Clewley G S, Fernandez T, et al. Comparative evaluation of the ExaVir Load version 3 reverse transcriptase assay for measurement of human immunodeficiency virus type 1 plasma load. J Clin Microbiol 2009 47: 3266-3270.
Matteucci D, Mazzetti P, Baldinotti F, Zaccaro L, Bendinelli M. The feline lymphoid cell line MBM and its use for feline immunodeficiency virus isolation and quantitation. Vet Immunol Immunopathol 1995 46:71-82.
Neogi U, Heylen E, Shet A, Chandy S, Shamsunder R, et al. Long-term efficacy of first line antiretroviral therapy in Indian HIV-1 infected patients: a longitudinal cohort study. PLoS One (2013) 8: e55421.
Pironti A1, Walter H, Pfeifer N, Knops E, Lübke N, Büch J, Di Giambenedetto S, Kaiser R, Lengauer T; EuResist Network Study Group. Determination of Phenotypic Resistance Cutoffs From Routine Clinical Data. J Acquir Immune Defic Syndr. 2017. 74(5):e129-e137.
Revets H, Marissens D, de Wit S, Lacor P, Clumeck N, Lauwers S, Zissis G. Comparative evaluation of NASBA HIV-1 RNA QT, AMPLICOR-HIV monitor, and QUANTIPLEX HIV RNA assay, three methods for quantification of human immunodeficiency virus type 1 RNA in plasma. J Clin Microbiol 1996; 34(5):1058-64
Roth, M. J., Tanese, N., Goff, S. P. Purification and characterization of murine retroviral reverse transcriptase expressed in Escherichia coli. J. Biol. Chem. 1985 260, 9326-9335.
Shao X, Malmsten A, Lennerstrand J, Sonnerborg A, Unge T, Gronowitz J S and Kallander C F R. Use of HIV-1 reverse transcriptase recovered from human plasma for phenotypic drug susceptibility testing. AIDS 2003, 17 1463-1471.
Taube R, Loya S, Avidan O, Perach M and Hizi A. Reverse transcriptase of mouse mammary tumour virus: expression in bacteria, purification and biochemical characterization. Biochem. J. 1998, 329, 579-587.
Usach I, Melis V, Peris J E. Non-nucleoside reverse transcriptase inhibitors: a review on pharmacokinetics, pharmacodynamics, safety and tolerability J Int AIDS Soc. 2013 Sep. 4; 16:1-14. doi: 10.7448/IAS.16.1.18567. Review

1. A method for assessing susceptibility of a virus to treatment with a drug inhibiting an enzyme of the wild-type virus comprising the steps:
a) Extracting the viral enzyme from a sample containing the virus;
b) Measuring the viral enzyme activity both in the absence of the drug and in the presence of the drug at a single predetermined concentration; and
c) Determining the virus' susceptibility to treatment with the drug from the relation between the enzyme activities in the presence of the drug and the absence of the drug.

2. A method for assessing whether a patient treated for a viral infection is in need of a change in drug therapy with a drug inhibiting a viral enzyme, due to the virus' resistance to the drug therapy, comprising assessing the virus' susceptibility to treatment with the drug, according to claim 1, wherein a susceptibility below a predetermined cut-off level is indicative of a need for a change in drug therapy.

3. A method for determining the load of an virus in a patient sample and said virus resistance to treatment with a drug inhibiting an enzyme of the wild-type virus, said method comprising the steps
a) Extracting the enzyme from a sample containing the virus;
b) Partitioning the extracted enzyme into at least two, a first and a second, aliquots;
c) Measuring the enzyme activity in said first aliquot in the absence of the drug and measuring the enzyme activity in said second aliquot in the presence of the drug at a single predetermined concentration;
d) Providing a series of standard enzyme activity values correlating enzyme activity to viral load;
e) Determining the viral load in the sample from the enzyme activity in the absence of the drug, based on the enzyme activity standard values; and
f) Determining the viral resistance to treatment with the drug from the relation between the enzyme activities in the presence of the drug and the absence of the drug, respectively.

4. The method according to claim 1, wherein the virus is a retrovirus and the enzyme is a reverse transcriptase packed into said retrovirus.

5. The method according to claim 4, wherein the retrovirus is a lentivirus, such as HIV, SIV, FIV; a β-retrovirus, such as JSRV or MMTV; a δ-retrovirus, such as BLV, HTLV-1 or HTLV-2; or a γ-retrovirus, such as PERV or MMuLV.

6. The method according to claim 4 or 5, wherein the drug is a Non Nucleoside Reverse Transcriptase Inhibitor (NNRTI) or a Nucleoside Reverse Transcriptase Inhibitor (NRTI).

7. The method according to claim 6, wherein the drug is a NNRTI drug selected from the group consisting of nevirapine, efavirenz, rilpivirine, etravirine, delavirdine, lersivirine, GSK 2248761, RDEA806, BILR 355 BS, calanolide A, MK-4965, MK-1439, MK-6186, doravirine, and elsulfavirine.

8. The method according to claim 6, wherein the drug is a NRTI drug selected from the group consisting of zidovudine, didasonine, zalcitabine, stavudine, lamivudine, abacavir, emtricitabine, tenofovir disoproxil fumarate, censavudine, MK-8591, adefovir, telbivudine and entecavir.

9. The method according to claim 1, wherein the predetermined drug concentration corresponds to the IC₅₀ value for a reference enzyme with known level of drug resistance.

10. The method according to claim 1, wherein the predetermined concentration corresponds to 10-50 times the IC₅₀ value for the wild-type enzyme for the drug, such as 15, 20, 25, 30, 35, 40, or 45 times the IC₅₀ value for the wild-type enzyme for the drug.

11. The method according to claim 1, wherein the sample is a blood sample, serum sample, plasma sample, a virus preparation from a cell culture, breast milk, saliva, semen, genital secretions, urine, intraperitoneal fluid or cerebrospinal fluid.

12. System for performing at least steps c)-f) of the method according to claim 3, comprising a computer and an apparatus for determining enzyme activity in a sample, said apparatus being configured to transmit enzyme activity values to said computer, and wherein the computer is configured to receive said enzyme activity values and a series of standard values correlating enzyme activity values to viral load, and is programmed to determine the viral load in the sample from the enzyme activity in the absence of the drug, based on the standard values correlating enzyme activity values to viral load; and to determine the virus' resistance to treatment with the drug from the relation between the enzyme activities in the presence of the drug and the absence of the drug.

13. System according to claim 12, wherein the apparatus for determining enzyme activity in a sample further comprises an apparatus for automated extraction of viral enzyme from a sample.
